(19) United States
(12) Patent Application Publication
(10) Pub. No.: US 2012/0045813 A1
(43) Pub. Date: Feb. 23, 2012

(54) GENE ENCODING A PROTEIN HAVING AN ABILITY TO ENHANCE A SELENATE REDUCTION ACTIVITY

(76) Inventors: Mitsuo Yamashita, Tokyo (JP); Michihiko Ike, Osaka (JP)

(21) Appl. No.: 13/132,362

(22) PCT Filed: Oct. 5, 2009
(86) PCT No.: PCT/JP2009/067332

§ 371 Date: Jun. 2, 2011

(30) Foreign Application Priority Data
Dec. 19, 2008 (JP) 2008-323235

(51) Int. Cl. C12P 3/00; C12N 9/48; C07H 21/04; C12N 9/14

(52) U.S. Cl. 435/168; 435/195; 435/212; 536/23.2

(57) ABSTRACT

According to the present invention, a protein having an ability to enhance a selenate reduction activity, a gene encoding it, and a method for selenate reduction using them are provided

### TECHNICAL FIELD

The present invention relates to a protein having an ability to enhance a selenate reduction activity, a gene encoding it, and a method for selenate reduction using them.

### BACKGROUND ART

Although selenium is a type of trace metal that is essential for a living organism, a water-soluble selenium compound (such as selenate or selenite) is toxic to the living organism. Since selenium is used in a wide range of applications, such as in a copying machine or for a coloring glass, it is important to secure a supply source thereof. In addition, an effect of the selenium compound present in wastewater and industrial waste on human health and the ecosystem are also becoming a problem. Although the methods such as a resin adsorption or electrochemical method have been examined as the methods for detoxifying and removing selenate, these methods have yet to be applied practically due to the problems relating to the efficiency, cost and other factors. In addition, selenium is unable to be recovered and reused by these methods.

The inventors of the present invention isolated a Gram-positive bacterium Bacillus selenatarsenatis strain SF-1 (to be referred to as “strain SF-1”) from a sludge of a glass factory with an aim of developing a method for biological treatment of a selenium compound (Patent Document 1, Non-Patent Document 1, Non-Patent Document 2). The strain SF-1 has an ability to efficiently reduce selenate to selenite, and further reduce selenite to elementary selenium. Since elementary selenium is insoluble in water and non-toxic, an use of this stain SF-1 has a potential to enable wastewater, etc., containing a selenium compound to be detoxified comparatively inexpensively as well as enable selenium to be recovered therefrom.

In order to treat more efficiently a selenium compound and recover selenium using a microorganism, it is necessary to identify the molecular mechanism involved in reduction of the selenium compound in addition to examining the treatment conditions and developing the equipments. However, any findings relating to an enzyme involved in reduction of the selenium compound and a gene encoding the enzyme have been hardly obtained. The inventors of the present invention analyzed the genes involved in reduction of the selenium compound (Non-Patent Document 3), and cloned a DNA fragment containing three open reading frames (ORFs) from the strain SF-1 (Non-Patent Document 4) wherein the DNA fragment demonstrates the ability to efficiently reduce selenate to selenite when introduced into Escherichia coli, in order to elucidate the mechanism by which the strain SF-1 reduces the selenium compound. However, these are only a portion of a group of genes involved in reduction of the selenium compound, and further analyses were required.
[Patent Document 1] Japanese Unexamined Patent Publication No. H9-248595
[Non-Patent Document 1] Fujita, M. et al., J. Ferment. Bioeng., 83:517-522 (1997)
[Non-Patent Document 2] Yamamura, S. et al., Int. J. Syst. Evol. Microbiol., 57:1060-1064 (2007)
[Non-Patent Document 3] Kuroda, M., et al., Abstract of Presentations of the 57th Annual Meeting of the Society for Biotechnology, Japan, 3A10-2 (2005)
[Non-Patent Document 4] Nagano, K., et al., Abstract of Presentations of the 60th Annual Meeting of the Society for Biotechnology, Japan, 1 Bp09 (2008)

### DISCLOSURE OF THE INVENTION

### Problems to be Solved by the Invention

An object of the present invention is to provide a protein having the ability to enhance the selenate reduction activity, a gene encoding it, and a method for selenate reduction using them.

### Means for Solving the Problems

The present invention relates to:

[1] a protein selected from the group consisting of the following:

(a) a protein consisting of the amino acid sequence of SEQ ID NO: 8,

(b) a protein that consists of an amino acid sequence in which one or more amino acids have been deleted, substituted or added in the amino acid sequence of SEQ ID NO: 8 and has an ability to enhance a selenate reduction activity in case of combining with a protein consisting of the amino acid sequence of SEQ ID NO: 10, and

(c) a protein that consists of an amino acid sequence having a sequence identity of 50% or more with the amino acid sequence of SEQ ID NO: 8 and has an ability to enhance a selenate reduction activity in case of combining with a protein consisting of the amino acid sequence of SEQ ID NO: 10;

[2] a protein selected from the group consisting of the following:

(a) a protein consisting of the amino acid sequence of SEQ ID NO: 10,

(b) a protein that consists of an amino acid sequence in which one or more amino acids have been deleted, substituted or added in the amino acid sequence of SEQ ID NO: 10 and has an ability to enhance a selenate reduction activity in case of combining with a protein consisting of the amino acid sequence of SEQ ID NO: 8, and

(c) a protein that consists of an amino acid sequence having a sequence identity of 60% or more with the amino acid sequence of SEQ ID NO: 10 and has an ability to enhance a selenate reduction activity in case of combining with a protein consisting of the amino acid sequence of SEQ ID NO: 8;

[3] a nucleic acid encoding the protein of [1];
[4] the nucleic acid of [3] selected from the group consisting of the following:

(a) a nucleic acid consisting of the nucleotide sequence of SEQ ID NO: 7, and

(b) a nucleic acid that hybridizes under the stringent conditions with a nucleic acid consisting of the nucleotide sequence of SEQ ID NO: 7 and encodes a protein having an ability to enhance a selenate reduction activity in case of combining with a protein consisting of the amino acid sequence of SEQ ID NO: 10;

[5] a nucleic acid encoding the protein of [2];
[6] the nucleic acid of [5] selected from the group consisting of the following:

(a) a nucleic acid consisting of the nucleotide sequence of SEQ ID NO: 9, and

(b) a nucleic acid that hybridizes under the stringent conditions with a nucleic acid consisting of the nucleotide sequence of SEQ ID NO: 9 and encodes a protein having an ability to enhance a selenate reduction activity in case of combining with the protein consisting of the amino acid sequence of SEQ ID NO: 8;

[7] a method for reduction of selenate, comprising the expressions of the protein of [1] and the protein of [2] in a host cell;
[8] the method of [7], wherein the expressions of the protein of [1] and the protein of [2] are carried out by introducing the nucleic acid of [3] and the nucleic acid of [4] into a host cell; and,
[9] the method of [7], further comprising the expressions of the protein consisting of the amino acid sequence of SEQ ID NO: 2, the protein consisting of the amino acid sequence of SEQ ID NO: 4, and the protein consisting of the amino acid sequence of SEQ ID NO: 6 in a host cell.

### Effects of the Invention

According to the present invention, a protein having an ability to enhance a selenate reduction activity, a gene encoding it, and a method for selenate reduction using them are provided.

### BRIEF DESCRIPTION OF THE DRAWINGS

FIG. 1 is a drawing indicating the location of three ORFs of mpoA (indicated with the number “3”), mpoB (indicated with the number “4”) and mpoC (indicated with the number “5”), the orientation of transcription/translation (indicated with arrows), and a Tn916 insertion site (indicated with “Tn916”) in a mutant strain deficient in the ability to produce elementary selenium.

FIG. 2 is a drawing indicating the location of two ORFS of dcpA (indicated with the number “3”) and mutT (indicated with the number “4”), the orientation of transcription/translation (indicated with arrows), and a Tn916 insertion site (indicated with “Tn916”) in a mutant strain deficient in the ability to produce elementary selenium.

FIG. 3 is a drawing indicating the constructions of the expression vectors for mpoA, mpoB and mpoC as well as dcpA and mutT: A: pGEM-mpoABC, B: pGEM-dcpAmutT, C: pGEM-dcpAmutT-mpoABC, D: pGEM-mpoABC-dcpAmutT.

FIG. 4 is a drawing indicating the effects of mpoA, mpoB and mpoC as well as dcpA and mutT on selenate reduction in Escherichia coli.

### BEST MODE FOR CARRYING OUT THE INVENTION

The term “selenium compound” used in the present specification refers to a compound containing selenium. Examples of the selenium compounds include selenate and selenite, etc. The term “elementary selenium” used in the present specification refers to selenium in the elemental state that has not formed a compound with other elements.

The term “selenate reduction activity” used in the present specification refers to an activity that reduces selenate to selenite. The term “selenatc reductase” used in the present specification refers to a protein that catalyzes the reduction of selenate to selenite. The term “selenite reduction activity” used in the present specification refers to an activity that reduces selenite to elementary selenium. The term “selenite reductase” used in the present specification refers to a protein that catalyzes the reduction of selenite to elementary selenium.

Bacillus selenatarsenatis strain SF-1 (strain SF-1) is known to have a selenate reduction activity and a selenite reduction activity (Patent Document 1, Non-Patent Document 1, Non-Patent Document 2). The selenate reduction activity can be measured by quantifying selenite produced from selenate. The selenite reduction activity can be measured by quantifying elementary selenium produced from selenite. The selenate reductase activity possessed by a protein encoded by a nucleic acid can be confirmed by the production of elementary selenium, for example, when this gene is introduced into a Escherichia coli known to have the selenite reduction activity (Bebien, M., et al., Microbiology, 148:3865-3872 (2002)) by observing red coloring of the colonies on the medium containing selenate.

The term “an ability to enhance a selenate reduction activity” or “a selenite reduction activity enhancing ability” used in the present specification refers to the ability to enhance the selenate reduction activity. A selenate reduction activity is enhanced in case that the activity has increased significantly compared with a control when selenite produced from selenate has been quantified. The ability to enhance the selenate reduction activity possessed by a protein encoded by a cloned nucleic acid can be confirmed by the level of elementary selenium produced when this gene is introduced into Escherichia coli containing a gene that encodes selenate reductase, by observing an increase in the intensity of red coloring of the colonies on the medium containing selenate.

An example of a selenate reductase includes that consists of proteins MpoA (SEQ ID NO: 2), MpoB (SEQ ID NO: 4) and MpoC (SEQ TD NO: 6) encoded by three open reading frames (ORFs) consisting of mpoA (SEQ ID NO: 1), mpoB (SEQ ID NO: 3) and mpoC (SEQ ID NO: 5) isolated from the strain SF-1 by the inventors of the present invention (Non-Patent Document 4). (Proteins MpoA, MpoB and MpoC are respectively named SrdB, SrdC and SrdA based on their functions as selenate reductases (srd) and their similarities to clusters of tetrathionate reductase genes from Salmonella typhimurium, and ORF encoding these proteins are respectively named srdB, srdC and srdA.) MpoA, MpoB and MpoC respectively have homology with known molybdopterin oxide reductase iron-sulfur-binding subunits (iron-sulfur clusters), molybdopterin oxide reductase membrane subunits (membrane-bound subunits) and molybdopterin dinucleotide-binding domains (catalyst sites retaining a phosphate group binding site).

MpoA, MpoB and MpoC have the similarity with tetrathionate reductase from Salmonella typhimurium (Hensel, M. et al., Mol. Microbiol., 32:275-287 (1999)). Tetrathione reductase gene from Salmonella typhimurium forms the clusters, and has the structure in which ttrC gene of a membrane-bound subunit and ttrB gene of a subunit containing iron-sulfur clusters are located upstream from ttrA gene that encodes a subunit of an activity center portion having a molybdopterin guanine dinucleotide cofactor. This positional relationship is extremely similar to the positional relationship of mpoA, mpoB and mpoC. This document states that ttrA, ttrB and ttrC are the structural genes of tetrathionate reductase. Likewise, mpoA, mpoB and mpoC obtained by the inventors of the present invention are thus suggested to constitute a structural gene of molybdopterin oxide reductase. In addition, the fact that mpoB encodes a membrane-bound subunit coincides with a previous report that the selenate reductase of the strain SF-1 is a membrane-bound type (Jpn. J. of Wat. Treat. Biol., 40:161-168 (2004)).

An example of a protein having an ability to enhance a selenate reduction activity is a combination of proteins DcpA (SEQ ID NO: 8) and MutT (SEQ ID NO: 10) encoded by two ORFs of dcpA (SEQ ID NO: 7) and mutT (SEQ ID NO: 9) isolated from the strain SF-1 by the inventors of the present invention. When these proteins are expressed in Escherichia coli together with a selenate reductase (for example, one consisting of MpoA, MpoB and MpoC), a selenate reduction activity is enhanced in comparison with the case of expressing only a selenate reductase.

The amino acid sequence of DcpA (SEQ ID NO: 8) has homology with various known diguanylate cyclase/phosphodiesterases. Diguanylate cyclase is an enzyme that catalyzes a synthesis of cyclic diguanylate (c-di-GMP) from two molecules of GTP, while phosphodiesterase is an enzyme that catalyzes a decomposition of c-di-GMP. In general, diguanylate cyclase/phosphodiesterase is known to contain a GGDEF (Gly-Gly-Asp-Glu-Phe) sequence and EAL (Glu-Ala-Leu) sequence, and the regions that contain these sequences are referred to as the GGDEF domain and EAL domain, respectively (Mendez-Ortiz, M. M. et al., J. Biol. Chem., 281:8090-8099 (2006)). In addition, the former is suggested to be responsible for synthesis of c-di-GMP, while the latter is suggested to be responsible for decomposition of c-di-GMP (Tamayo, R. et al., Infection and Immunity, 76:1617-1627 (2008)). Although a GGDEF sequence can be found in DcpA (SEQ ID NO: 8, positions 238 to 242), an EAL sequence cannot be found. Thus, it is possible that DcpA only has a diguanylate cyclase activity responsible for synthesis of c-di-GMP.

The amino acid sequence of MutT (SEQ ID NO: 10) has homology with various known proteins of the MutT/nudix (nucleoside diphosphates linked to other moieties, X) family. The proteins of the MutT/nudix family are the generic term for the enzymes that catalyze a hydrolysis of nucleoside diphosphates bound to other molecules, while MutT is an enzyme that catalyzes a reaction that forms GMP by decomposing GTP.

Although the mechanism by which DcpA and MutT enhance the selenate reduction activity is unclear, in considering that the selenate reductase obtained from the strain SF-1 has homology with the molybdopterin oxide reductase containing molybdopterin as a cofactor, and that molybdopterin is synthesized from GTP (Cell Mol. Life. Sci., 62:2792-2810 (2005)), it is possible that DcpA and MutT are involved in synthesis of cofactors of the selenate reductase. In addition, MpoA and MpoC have the high degree of homology with Tat (Twin-arginine translocation) pathway signals. The Gram-positive bacterium Bacillus subtilis, is known to have the Sec pathway and the Tat pathway as a protein secretory pathway. In the Sec pathway, a structure of a transported protein is unfold when passing through the cell membrane. In the Tat pathway, a protein folded in the cytoplasm passes through the cell membrane while remaining folded. The protein containing a cofactor such as molybdopterin is said to be transported via this Tat pathway (van Dijil, J. M. et al., J. Biotechnol., 98:243-254 (2002)). This also suggests the possibility that the selenate reductase from the strain SF-1 contains a cofactor.

The inventors of the present invention reported that a selenate reduction activity is demonstrated when the three ORFs of mpoA, mpoB and mpoC are introduced into Escherichia coli (Non-Patent Document 4). On the basis thereof; the proteins encoded by these ORFs were suggested to be sufficient for reducing selenate. Thus, the finding that a selenate reduction activity is further enhanced by additionally introducing dcpA and mutT was unexpected. In the strain SF-1, since a selenate reduction activity is lost even in case that the transposon Tn916 is inserted into either the region encoding mpoA, mpoB and mpoC or the region encoding dcpA and mutT, the genes of both these regions are considered to be required for a selenate reduction activity in the strain SF-1. Although the reason why a selenate reduction activity is observed in Escherichia coli in the absence of dcpA and mutT is unclear, it is possible that the proteins playing as the functional substitutes for dcpA and mutT are present in Escherichia coli used as a host.

The sequence identity between the amino acid sequence of SEQ ID NO: 8 and the known sequence indicating the highest degree of homology (diguanylate cyclase with PAS/PAC sensor [Geobacillus sp. G11MC16], GenBank Accession No. ZP_{—}03149864) is 48%, while the sequence identity between the amino acid sequence of SEQ ID NO: 10 and the known sequence indicating the highest degree of homology (MutT/nudix family protein, putative [Bacillus cereus G92411], GenBank Accession No. ZP_{—}00238672) is 56%. The BLAST program (http://www.ncbi.nlm.nih gov/blast/Blast.cgi) was used to calculate an identity of amino acid sequences. Combinations of the proteins that consist of an amino acid sequence having the higher sequence identity with the amino acid sequence of SEQ ID NO: 8 or 10 than these sequences and have the ability to enhance the selenate reduction activity in case of combining with the protein of the amino acid sequence of SEQ ID NO: 8 or 10, can also be used preferably in the present invention. Such sequence identity is identity of, for example, 50%, preferably 70%, more preferably 80% and even more preferably 90%, with the amino acid sequence of SEQ ID NO: 8 and identity of, for example, 60%, preferably 70%, more preferably 80%, and even more preferably 90% with the amino acid sequence of SEQ ID NO: 10.

In addition, in the present invention, a combination of proteins can also be used that consist of an amino acid sequence in which one or more of the amino acids in the amino acid sequence of SEQ ID NO: 8 or 10 is deleted, substituted or added, and have the ability to enhance the selenate reduction activity in case of combining with a protein consisting of the amino acid sequence of SEQ ID NO: 10 or 8.

In the present invention, a nucleic acid is used that encodes a protein having the ability to enhance the selenate reduction activity as described above. In one embodiment, this nucleic acid is a nucleic acid consisting of the nucleotide sequence of SEQ ID NO: 7 or 9. In another embodiment, the nucleic acid of the present invention is a nucleic acid encoding a protein that hybridizes under the stringent conditions with a nucleic acid consisting of the nucleotide sequence of SEQ ID NO: 7 or 9 and has the ability to enhance the selenate reduction activity in case of combining with the protein consisting of the amino acid sequence of SEQ ID NO: 10 or 8. The term “stringent conditions” used in the present specification refers to the stringent hybridization conditions. Such conditions are described in, for example, Sambrook, J. et al. (eds), Molecular Cloning: A Laboratory Manual, Second Edition, Cold Spring Harbor Laboratory Press (1989). An example of the stringent conditions in case of using a long probe of 100 or more nucleotides includes incubating in 6×SSC, 0.5% sodium dodecyl sulfate (SDS), 5×Denhardt's reagent, denaturation-fragmented salmon sperm DNA at 100 μg/mL, at 68° C. and washing in 2×SSC, 0.1% SDS at room temperature (decreasing the SSC concentration to 0.1 and/or raising the temperature to 68° C.).

In the selenate reduction method of the present invention, a protein having the ability to enhance the selenate reduction activity is expressed in the host cells. Although any cells can be used for the host cells, any bacteria able to survive in the presence of a selenium compound are preferable. In one embodiment, an expression of protein having an ability to enhance a selenate reduction activity is carried out by introducing a nucleic acid that encodes this protein into the host cells. The bacteria for which the recombinant DNA technology has been established are used preferably as a host in order to achieve this objective. Examples of such bacteria include, but are not limited to, Escherichia coli and Bacillus subtilis, etc. A vector capable of replicating in the selected host cells is used to introduce a nucleic acid. A vector from a plasmid, bacteriophage or virus and the like can be used. A sequence responsible for starting and stopping a transcription of an interested protein (such as a promoter or terminator), and a sequence required to start a translation (such as a ribosome binding site) are contained in the vector containing the nucleic acid. A person with ordinary skill in the art can select these sequences that are suitable for the host cells. For example, the promoter present originally upstream from a sequence that encodes a protein having an ability to enhance a selenate reduction activity can be used in case that the promoter functions in the host cells. Alternatively, an interested gene can be located and expressed under the control of a different promoter that functions in the host cells. In case that the host cells do not have a selenate reductase, the selenate reductase may be further expressed in the host cells.

Although the following provides a detailed explanation of the present invention through Examples, the present invention is not limited to these examples.

### EXAMPLES

### Reference Example 1

### Isolation of a Mutant Strain Deficient in an Ability to Produce Elementary Selenium

Transposon Tn916 which retains a tetracycline resistance gene (Scott, J. R. et al., Annu. Rev. Microbiol., 49:367-397 (1995) was introduced into a spontaneous streptomycin-resistant mutant strain (to be referred to as “strain Sm^{r}”) of Bacillus selenatarsenatis strain SF-1 (JCM14380, DSM18680) from Enterococcus faecalis strain CG110 (to be referred to as “strain CG110”) by conjugational transfer in order to obtain a mutant strain deficient in the ability to produce elementary selenium. Tn916 was then inserted into the introduced bacterial genome. Thus, as a result of disrupting a gene involved in reduction of selenate or reduction of selenate by inserting Tn916, a strain deficient in the ability to produce elementary selenium from selenate may be present among Tn916 introducing strains.

Strain Sm^{r} which was shake-cultured for 20 hours at 37° C. in 3 mL of TSB (Trypticase Soy Broth) medium (containing 17.0 g/L of casein, 3.0 g/L of soybean peptone, 2.5 g/L of dextrose, 5.0 g/L of sodium chloride and 2.5 g/L of dipotassium phosphate) containing 500 μg/mL of streptomycin, was centrifuged at 7,000 rpm and 4° C. to collect the bacteria. Strain CG110 was cultured for 20 hours at 37° C. on LB agar medium (containing 10 g/L of bactotrypsin, 5 g/L of yeast extract and 5 g/L of sodium chloride). A suspension of the Sm^{r} srain was added to this plate, the two species of bacteria were mixed, and then cultured overnight at 37° C. The developing bacteria were suspended in 10 mL of TSB medium and 200 μL of a 100-fold dilution thereof were inoculated into a layered selenate selective medium (pouring and solidifying LB agar medium containing 500 μg/mL of streptomycin, 10 μg/mL of tetracycline and 0.5 mM selenate followed by pouring and solidifying LB agar medium containing an equal amount of streptomycin at 500 μg/mL and tetracycline at 10 μg/mL) followed by incubating overnight at 37° C. A Tn916-introducing strain was obtained as a colony showing a resistance to tetracycline and streptomycin.

The resultant plates were incubated at 30° C. The strain having the ability to produce elementary selenium forms a red colony, but the strain deficient in the ability to produce elementary selenium forms a white colony. The white, relatively small colony was selected (primary screening). After aerobically culturing this colony overnight at 37° C. in on LB agar medium containing 500 μg/mL of streptomycin, 10 μg/mL of tetracycline and 1 mM sodium selenate, the colony was anaerobically cultured for 2 days at 30° C. using an AnaeroPouch KENKI (Mitsubishi Gas Chemical). After culturing, the strain that exhibit decreased red color or not red color, which indicates the ability to produce elementary selenium, was obtained (secondary screening).

### Reference Example 2

### Determination of DNA Sequence Surrounding the Tn916 Insertion Site by the Inverse PCR and LA PCR

Genomic DNA was prepared from the strain deficient in the ability to produce elementary selenium obtained in Reference Example 1 using the AquaPure Genomic DNA Kit (BIO-RAD). After digesting this genomic DNA with a suitable restrict enzyme, the resulting DNA was subjected to self-ligation using T4 DNA ligase. By then carrying out a polymerase chain reaction using this reaction mixture as a template and using the Tn916-specific primers, DNA surrounding the Tn916 insertion site was amplified and the nucleotide sequence thereof was determined. By DNA amplification using the LA PCRO in vitro cloning kit (Takara Bio) using the primers synthesized based on the nucleotide sequence obtained in this manner and using the genomic DNA of the strain SF-1 in which Tn916 was not inserted as a template, DNA surrounding the site where Tn916 was inserted was amplified and the nucleotide sequence thereof was determined Takara LA Taq or PrimeSTAR GXL DNA Polymerase (Takara Bio) provided with the kit was used as a DNA polymerase in LA PCR.

As a result of searching for open reading frames (ORFs) in the nucleotide sequences determined according to the above procedures, and further comparing the amino acid sequence encoded therein with the amino acid sequences of known proteins, two regions were identified which encode the proteins having the possibility of being involved in reduction of the selenium compounds.

### Reference Example 3

### Analysis of mpoABC Operon

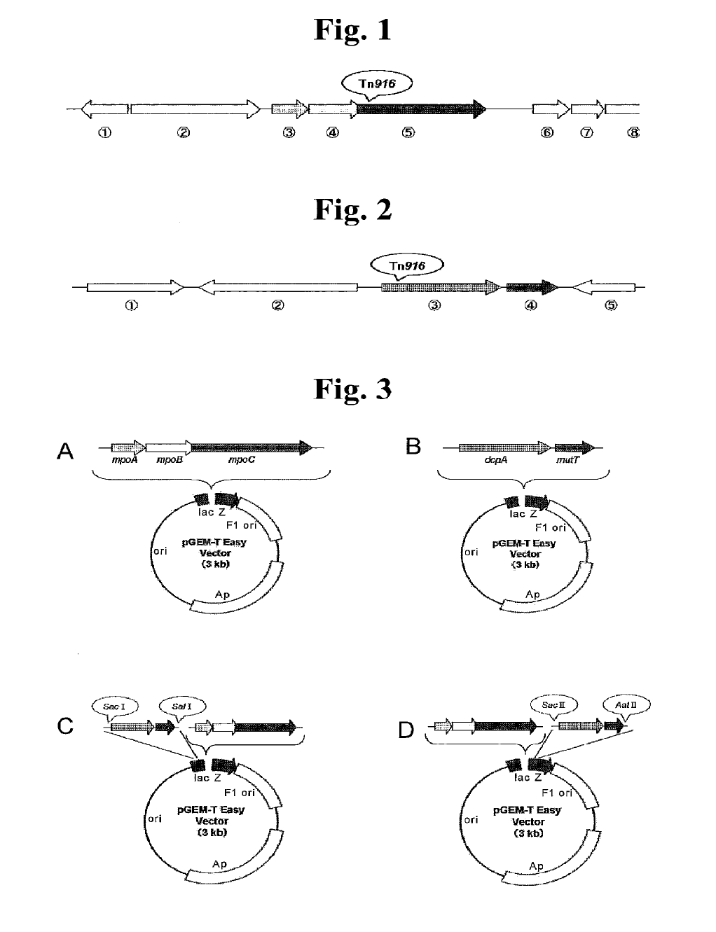

A region was found using the procedure described in Reference Example 2 that contained three ORFs encoding the proteins having homology with a known molybdopterin oxide reductase iron-sulfur-binding subunit (iron-sulfur cluster), molybdopterin oxide reductase membrane subunit (membrane-bound subunit) and molybdopterin dinucleotide-binding region (phosphate group binding site). These ORFs were present in the above order from upstream to downstream and in the same orientation (indicated with numbers [3], [4] and [5] in FIG. 1). These were respectively named mpoA, mpoB and mpoC. The nucleotide sequences of mpoA, mpoB and mpoC are respectively shown in SEQ ID NO: 1, 3 and 5, while the amino acid sequences of proteins MpoA, MpoB and MpoC encoded by these nucleotide sequences are respectively shown in SEQ ID NO: 2, 4 and 6. Since the distance between the stop codon of mpoA and the start codon of mpoB is extremely adjacent at 17 bp, and mpoB and mpoC overlap by about 40 bp, it was thought that these three ORFs form an operon in which the transcriptions is started by a promoter-like region located upstream from mpoA and stopped by a terminator-like region present downstream from mpoC. Furthermore, Tn916 was inserted in the mpoC region of the mutant strain deficient in the ability to produce elementary selenium obtained in Reference Example 1 (indicated with “Tn916” in FIG. 1).

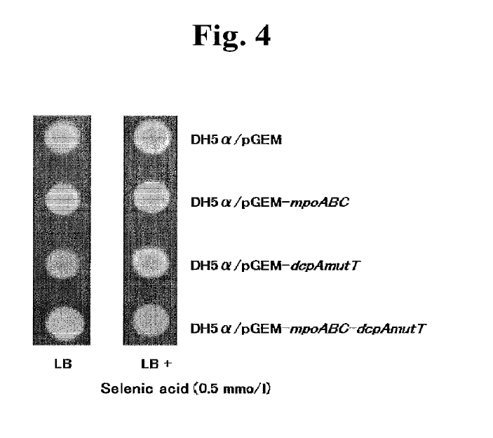

The region containing the promoter and the three ORFs of mpoA, mpoB and mpoC was amplified using primers OPERON1F (SEQ ID NO: 11) and OPERON1R (SEQ ID NO: 12), and inserted into a multi-cloning site of the TA cloning vector pGEM®-T Easy Vector (Promega) to obtain a plasmid pGEM-mpoABC (FIG. 3A). Escherichia coli D5α competent cells were then transformed using this plasmid. The resulting transformed strain DH5α/pGEM-mpoABC was inoculated on LB medium containing 0.5 mmol/L of selenate together with a control strain DH5α/pGEM transformed with a plasmid not containing mpoA, mpoB or mpoC. A medium not containing selenate was used as a control. Furthermore, since Escherichia coli inherently possesses the ability to reduce selenite (Bebien, M. et al., Microbiology, 148:3865-3872 (2002)), it is able to produce elementary selenium on the medium containing selenate if the transformant has the ability to reduce selenate to selenite, and the resulting colonies become to be a red color. As a result, since the DH5α/pGEM-mpoABC strain exhibited the red color, it was demonstrated to show the activity that reduces selenate to selenite in Escherichia coli introduced with the three ORFs (DH5α/pGEM-mpoABC in FIG. 4).

### Example 1

### Analysis of dcpAmutT Operon

A region was found using the procedure described in Reference Example 2 that contains two ORFs encoding the proteins having homology with a known diguanylate cyclase/phosphodiesterase and MutT nudix family protein. These ORFs were present in the above order from upstream to downstream and in the same orientation (indicated with numbers [3] and [4] in FIG. 2). These ORFs were respectively named dcpA and mutT. The nucleotide sequences of dcpA and mutT are respectively shown in SEQ ID NO: 7 and 9, while the amino acid sequences of proteins DepA and MutT encoded by these nucleotide sequences are respectively shown in SEQ ID NO: 8 and 10. Since the distance between the stop codon of dcpA and the start codon of mutT is extremely adjacent at about 30 bp, it was thought that these two ORFs form an operon in which the transcription is started by a promoter-like region located upstream from dcpA and stopped by a terminator-like region located downstream from mutT. Furthermore, Tn916 was inserted into the dcpA region in the mutant strain deficient in the ability to produce elementary selenium obtained in Reference Example 1 (indicated with “Tn916” in FIG. 2).

A region containing a promoter and the two ORFs of dcpA and mutT, was amplified using primers ALLGGDEFFW (SEQ ID NO: 13) and ALLGGDEFRV (SEQ ID NO: 14), and inserted into a multi-cloning site of the TA cloning vector pGEM-T Easy Vector® to obtain a plasmid pGEM-dcpAmutT (FIG. 3B). Escherichia coli DH5α competent cells were then transformed using this plasmid. The resulting transformed strain DH5α/pGEM-dcpAmutT was inoculated on the medium containing 0.5 mmol/L of selenate together with a control strain DH5α/pGEM transformed with a plasmid not containing dcpA, mutT. As a result, since neither of strains exhibited a red color, the two ORFs were demonstrated to not show the activity that directly reduces selenate to selenite (DH5α/pGEM-dcpAmutT in FIG. 4).

Next, a region containing the two ORFs of dcpA and mutT, was amplified using primers UPALLGGDEFFW (SEQ ID NO: 15) and UPALLGGDEFRV (SEQ ID NO: 16) or primers DOWNALLGGDEFFW (SEQ ID NO: 17) and DOWN ALLGGDEFRV (SEQ ID NO: 18) to obtain plasmids pGEM-dcpAmutT-mpoABC and pGEM-mpoABC-dcpAmutT inserted in the same orientation upstream or downstream from mpoA, mpoB and mpoC in the plasmid pGEM-mpoABC obtained in Reference Example 3 (FIGS. 3C and 3D). When the strain DH5α/pGEM-mpoABC-dcpAmutT containing pGEM-mpoABC-dcpAmutT was inoculated in the similar manner on the medium containing selenate, a more intense red color was observed than the strain DH5α/pGEM-mpoABC (DH5α/pGEM-mpoABC-dcpAmutT of FIG. 4). Similar results were obtained for a strain introduced pGEM-dcpAmutT-mpoABC (data not shown). In this manner, the two genes dcpA and mutT were demonstrated to have the ability to enhance the selenate reduction activity shown by mpoA, mpoB and mpoC in Escherichia coli.

### INDUSTRIAL APPLICABILITY

According to the present invention, a protein having the ability to enhance selenate reduction activity, a gene encoding it, and a method for a selenate reduction using them are provided.

### SEQUENCE LISTINGS

1. A protein selected from the group consisting of the following:
(a) a protein consisting of the amino acid sequence of SEQ ID NO: 8,
(b) a protein that consists of an amino acid sequence in which one or more amino acids have been deleted, substituted or added in the amino acid sequence of SEQ ID NO: 8 and has an ability to enhance a selenate reduction activity in case of combining with a protein consisting of the amino acid sequence of SEQ ID NO: 10, and
(c) a protein that consists of an amino acid sequence having a sequence identity of 50% or more with the amino acid sequence of SEQ ID NO: 8 and has an ability to enhance a selenate reduction activity in case of combining with a protein consisting of the amino acid sequence of SEQ ID NO: 10.

2. A protein selected from the group consisting of the following:
(a) a protein consisting of the amino acid sequence of SEQ ID NO: 10,
(b) a protein that consists of an amino acid sequence in which one or more amino acids have been deleted, substituted or added in the amino acid sequence of SEQ ID NO: 10 and has an ability to enhance a selenate reduction activity in case of combining with a protein consisting of the amino acid sequence of SEQ ID NO: 8, and
(c) a protein that consists of an amino acid sequence having a sequence identity of 60% or more with the amino acid sequence of SEQ ID NO: 10 and has an ability to enhance a selenate reduction activity in case of combining with a protein consisting of the amino acid sequence of SEQ ID NO: 8.

3. A nucleic acid encoding the protein according to claim 1.

4. The nucleic acid according to claim 3 selected from the group consisting of the following:
(a) a nucleic acid consisting of the nucleotide sequence of SEQ ID NO: 7, and
(b) a nucleic acid that hybridizes under the stringent conditions with a nucleic acid consisting of the nucleotide sequence of SEQ ID NO: 7 and encodes a protein having an ability to enhance a selenate reduction activity in case of combining with a protein consisting of the amino acid sequence of SEQ ID NO: 10.

5. A nucleic acid encoding the protein according to claim 2.

6. The nucleic acid according to claim 5 selected from the group consisting of the following:
(a) a nucleic acid consisting of the nucleotide sequence of SEQ ID NO: 9, and
(b) a nucleic acid that hybridizes under the stringent conditions with a nucleic acid consisting of the nucleotide sequence of SEQ ID NO: 9 and encodes a protein having an ability to enhance a selenate reduction activity in case of combining with a protein consisting of the amino acid sequence of SEQ ID NO: 8.

7. A method for reduction of selenate, comprising:
(i) expressing of the protein according to claim 1 in a host cell, and
(ii) expressing a second protein in the host cell, wherein the second protein is selected from the group consisting of the following:
(a) a protein consisting of the amino acid sequence of SEQ ID NO: 10,
(b) a protein that consists of an amino acid sequence in which one or more amino acids have been deleted, substituted or added in the amino acid sequence of SEQ ID NO: 10 and has an ability to enhance a selenate reduction activity in case of combining with a protein consisting of the amino acid sequence of SEQ ID NO: 8, and
(c) a protein that consists of an amino acid sequence having a sequence identity of 60% or more with the amino acid sequence of SEQ ID NO: 10 and has an ability to enhance a selenate reduction activity in case of combining with a protein consisting of the amino acid sequence of SEQ ID NO: 8.

8. A method for reduction of selenate, comprising:
(i) introducing a nucleic acid that encodes the protein according to claim 1 into a host cell, and
(ii) introducing a second nucleic acid that encodes a second protein into the host cell, wherein the second protein is selected from the group consisting of the following:
(a) a protein consisting of the amino acid sequence of SEQ ID NO: 10,
(b) a protein that consists of an amino acid sequence in which one or more amino acids have been deleted, substituted or added in the amino acid sequence of SEQ ID NO: 10 and has an ability to enhance a selenate reduction activity in case of combining with a protein consisting of the amino acid sequence of SEQ ID NO: 8, and
(c) a protein that consists of an amino acid sequence having a sequence identity of 60% or more with the amino acid sequence of SEQ ID NO: 10 and has an ability to enhance a selenate reduction activity in case of combining with a protein consisting of the amino acid sequence of SEQ ID NO: 8.

9. The method according to claim 7, further comprising the expressions of a protein consisting of the amino acid sequence of SEQ ID NO: 2, a protein consisting of the amino acid sequence of SEQ ID NO: 4, and a protein consisting of the amino acid sequence of SEQ ID NO: 6 in a host cell.


SEQUENCE LISTING (19 sequences)

SEQ ID NO: 1 - DNA, 879 bp, Bacillus selenatarsenatis SF-1
atgggttcaaaagagacaaaaaacacatcaagaagagactttcttataaagggggcaggtgcagcagccttaggtgccggagcatttgctattagtcaagtccctttattagaaaaattggcttcagcaaatgaggatacagtcaaagacctgctcccttttcctgaattaattgaatcagatgaaattattattagaatgcaaaacgatgttaggagggcgctaaaaaaaccattaaatgaaatccaatggatcatggtgattgacttaaagaaatgtgtgggttgttcttcctgtactgtggcatgcgtttcagagaatgtattgcctcccggtgttgtttaccgtccggttatcgaggaagagattgggacatttcctaatgtgacaaagaagtttaccccaagaccatgtatgcaatgtgaacatccaccatgtacaaaggtttgcccgattggtgcaacttataaaagtgaagatggaattgtggcaattgattatgataagtgtatcggttgtcgctattgcattactgcttgcccgtatggtgcgagaacattcgactggggtgagtatcatactgagaatacacctgaaatcatgcaatatgaaaaagagccaaactatgagtatggagtagagcgagtaagagaaaagaaaaattcaccaattggaaatgcaagaaagtgtcatttctgtaaacaccgacttcataaaggaatgctgtctatgtgtgtaacaacctgcattggtagagcaacatatattggtgataagaatgatcctgagagcttggtagcagaattaatagcttcacctcgagttatgagattgaaagaagaattaggtactgaaccaaatgtatattacttaacataa

SEQ ID NO: 2 - protein, 292 aa, Bacillus selenatarsenatis SF-1
MGSKETKNTSRRDFLIKGAGAAALGAGAFAISQVPLLEKLASANEDTVKDLLPFPELIESDEIIIRMQNDVRRALKKPLNEIQWIMVIDLKKCVGCSSCTVACVSENVLPPGVVYRPVIEEEIGTFPNVTKKFTPRPCMQCEHPPCTKVCPIGATYKSEDGIVAIDYDKCIGCRYCITACPYGARTFDWGEYHTENTPEIMQYEKEPNYEYGVERVREKKNSPIGNARKCHFCKHRLHKGMLSMCVTTCIGRATYIGDKNDPESLVAELIASPRVMRLKEELGTEPNVYYLT

SEQ ID NO: 3 - DNA, 1281 bp, Bacillus selenatarsenatis SF-1
atgttaaaaaaattatattttacagtgttatcatttattgcaattgttggtgtaataagcctttatataagattaagtgaaggtatgaaaatgtctgcacttactagttattccagctggggcttatggattgtattctatatatactttataggtctatcggcaggatcatttcttctatccacaatggtctatgtctttaatatgaaacaatttgagcgaattggaaagcttgcgctatttactgcatttttttccttaatggccggattgctcttcgtactaattgatttaggccaccctgaaagattttggcacactttagtttataggcaaccgaattcaattctatcttgggaaatccaattttacttaatttatatgctactaattgtggcagaaatttggtttttaatgagagaagaattcgcacaaagggcccagtctacaaaaggattaaccaaactgtttaacagaaccttaacactaggatataaggttcctaacagtcaacaaaagcttgagtttcatagagaacaaagtcataaatggatgaaaatcttggggattgctggaattccaacggcagttggggtccacggaggaactggctcattatttggtgtagtaatggcaaaagaatattggttctcaggacttactcctatcattttcttagtttccgctttagtctctggtgcggcattaatgttgttcttgtactcgttttttggtggtgcaaaaaagaacggggattctctcttaaaggaattaggaaccctactaacattatttattggtatcgatctcttgttaatgatcgcagaatttctaattggcctatataatcctatacaccatgaaagaatgacatttaatgaaatcctctttggggataggtggtttatattttggattggacaaatcggaatggtaattatattgccaattctccttattaccataagtaaagggaagaggctattgatgggcttagctggactttctgtggttttaggtattgtatgtgttagatggattctcgtaatcccggcatatgtagcgccgcattttgatggtttagatagcgcctataattcatcccgtttactttacgaatattctcccaatttgcttgagtggtcttcaagcgtaggattaatcggtattgtgattttattgtttagtataactgtccaattagtaccagtgttcaataagcaaaaggaggtacatcaaactcatggaaaaccaacaccagaaattcatatcaaggcgtaa

SEQ ID NO: 4 - protein, 426 aa, Bacillus selenatarsenatis SF-1
MLKKLYFTVLSFIAIVGVISLYIRLSEGMKMSALTSYSSWGLWIVFYIYFIGLSAGSFLLSTMVYVFNMKQFERIGKLALFTAFFSLMAGLLFVLIDLGHPERFWHTLVYRQPNSILSWEIQFYLIYMLLIVAEIWFLMREEFAQRAQSTKGLTKLFNRTLTLGYKVPNSQQKLEFHREQSHKWMKILGIAGIPTAVGVHGGTGSLFGVVMAKEYWFSGLTPIIFLVSALVSGAALMLFLYSFFGGAKKNGDSLLKELGTLLTLFIGIDLLLMIAEFLIGLYNPIHHERMTFNEILFGDRWFIFWIGQIGMVIILPILLITISKGKRLLMGLAGLSVVLGIVCVRWILVIPAYVAPHFDGLDSAYNSSRLLYEYSPNLLEWSSSVGLIGIVILLFSITVQLVPVFNKQKEVHQTHGKPTPEIHIKA

SEQ ID NO: 5 - DNA, 3147 bp, Bacillus selenatarsenatis SF-1
atggaaaaccaacaccagaaattcatatcaaggcgtaattttattaaaacaagtgctttgttgggtggaacagcattcttgggaacgggactgcccaatataaaaaagacctattcaaaagaattggattatgttgggaattttgaatatccacttgcaaagccagaaaacatactttactcagcatgtttacaatgtacagttgcctgcagtataaaggtcaaaatcaataatggtgtttgtatgaaaatagatggcaatccatacagtgcaatgaacttaggggaaaatttaccttatgatttatcaccaaaagaagcagtaagcattgatggtaagctttgtccaaaaggtcaagcgggaatacaacatgcatatgatccctacagacttagaaaagtaataaaaagagatggtcctagaggttctggaaagtggaaaaccattccttatgaccaagcaatagatgaaattgtcaatggtgggaacatatttaaagatatcggtgaaaatcaaaatgttgaaggtttaaaggatatttttgttctaaaagatccaaaggttgcaaaagcaatggctgacgacgttacaaaaattagaaaaaaagaaatgactgttgatgaatttaaagcaaagcataaagataatttagatgtgttaatagacccgaaccatccagatcttggaccaaaaaataatcaatttttattccaggtcggtcgtatccataatgggcgaatagaatttacaaaaaggttcgtaaatgactcatttggtagcgtaaactggattgaaaagacgacattgtgtggtcagactagtaataaagcttgggtacattcaacaagagagtatcttgaaggaaagtggacgggaggaattaaatctccgcgacctgatcatagaaacactgaatttttattggtttttggatcaattgtatttgaagcaaactacgggccagtacaggaaactgaaccaatcactgaagggttggaaagtggaaggttaaagattgcagtagtagatccacgattgactaaagtcgcttcaaaggcttggaaatgggtaccgataaagcctgggaatgatgcagcttttgctcttggtatgattcgatggattattgagaatgaaagatatgatacaaagtttctacaaaatgcaactagactggcagcgacaaactctgatgaaccatcttacagtaatgcgacctatttagtaaaagtcgaaaaggacggtagagctgcaaagcatttaagagctaatgaaattggcataggcacagaaaaagaatttgtcgtaataagtaatggtaaaccagctgcggttgatccagaaaacagcaatctagcaatacaaggtgagctatttgttgataccaacttagaaggaattaaggtgaagtctcctctacaactaataaaggaagaagcatactcaaaggaattggctgaatgggcagaattatctggagcaaaacaaaaagatattgaggatatttctaaagagtttacatctcatggaaaaaaggcagcagtagaattttatcgaggggctatcaaacatactaatggttggtataatggtcaggcgttaatagtgctaaatcatctaattggtaatatgaattggcaaggtggaatttctaatactggcggtggttggtcttatattggagataaagaggggcaaccatatccaatggcaaacctacacccaaataaattatctaaatttggagtaccaataacaaaagaaggttggaaatacgaagaatcaactttgtttgaaggatatccagctaagcgtccttggtatccgtttagtggtaacgtggctcaggagacttggccgtccatttcagacggatatccatataaaattaaagctgccctaatttcatcccattcaccaatgtattcacttcctggtggtcatgtacagctaaagacactattagatactgaaaaagtaccattattaattgctagtgatattgtaattggtgattcaagtcaatatgttgattatattttcccagatttaacttatttagaaagatgggcaacgccaggacaatctcaccatatccgggttaaagtgaatcaagttagacagccagtaattgccccattaactgagaacgcaacagtatttggagaggaagttccaatatctttagaagctttaatgatgtccatatctgagaaagtcggcttatctggatttggtaaagacgcattcgggccaggtaaagatctaaatagaatggaagatttctatctaaaactagttgctaatattgcatctgggaataagcaaggtgaattagttaaagctgcagattcagaagaaatggaattatttaagtcttcacatagacatctcccaaagacagtttatgatattgaaaaatggcaacaaactctcacttctgatgagtggaaaagagtagtttatgtattaaaccgtggtggccgttttgcatcaagtgatgacgcttatgaaggctcgtttattaaaaagaaaatccctggtcttgcaagattatatttggaagagattgctgattctcgtaattccatttcaggtaattactttacaggttatccaaagtaccttccaattatggacatggcagaaaacttgataaatgatgaaggagaattccacctcattactaacaaagaggtctttggtactcagtcaagaactataactaactattgggcacaactagcgttacaaccagaaaactttgtcgtattaaatagtgtggatgcaaagaaattaaaggtagataatggcgataaggtcttagtaacatcaacgagcaatccaaaaggggaacacttaattgcagcggatgaaagtagaccaaccattggtaaggtaaaaatcgttgaaggtattcgccctggtgttgttgcaatttcaacgcattatggtcattggggttatggtgcaagagatattcaaatcgatggagaagttgtaaaaggtgaggaagtaagaggtttaggaatacatcctaatccactgtttaggctagatgacaatttgagaggaacaacccttagtgatccaattggcggaagtgcatcttattatgacactagagtaaatattcaaagagtttaa

SEQ ID NO: 6 - protein, 1048 aa, Bacillus selenatarsenatis SF-1
MENQHQKFISRRNFIKTSALLGGTAFLGTGLPNIKKTYSKELDYVGNFEYPLAKPENILYSACLQCTVACSIKVKINNGVCMKIDGNPYSAMNLGENLPYDLSPKEAVSIDGKLCPKGQAGIQHAYDPYRLRKVIKRDGPRGSGKWKTIPYDQAIDEIVNGGNIFKDIGENQNVEGLKDIFVLKDPKVAKAMADDVTKIRKKEMTVDEFKAKHKDNLDVLIDPNHPDLGPKNNQFLFQVGRIHNGRIEFTKRFVNDSFGSVNWIEKTTLCGQTSNKAWVHSTREYLEGKWTGGIKSPRPDHRNTEFLLVFGSIVFEANYGPVQETEPITEGLESGRLKIAVVDPRLTKVASKAWKWVPIKPGNDAAFALGMIRWIIENERYDTKFLQNATRLAATNSDEPSYSNATYLVKVEKDGRAAKHLRANEIGIGTEKEFVVISNGKPAAVDPENSNLAIQGELFVDTNLEGIKVKSPLQLIKEEAYSKELAEWAELSGAKQKDIEDISKEFTSHGKKAAVEFYRGAIKHTNGWYNGQALIVLNHLIGNMNWQGGISNTGGGWSYIGDKEGQPYPMANLHPNKLSKFGVPITKEGWKYEESTLFEGYPAKRPWYPFSGNVAQETWPSISDGYPYKIKAALISSHSPMYSLPGGHVQLKTLLDTEKVPLLIASDIVIGDSSQYVDYIFPDLTYLERWATPGQSHHIRVKVNQVRQPVIAPLTENATVFGEEVPISLEALMMSISEKVGLSGFGKDAFGPGKDLNRMEDFYLKLVANIASGNKQGELVKAADSEEMELFKSSHRHLPKTVYDIEKWQQTLTSDEWKRVVYVLNRGGRFASSDDAYEGSFIKKKIPGLARLYLEEIADSRNSISGNYFTGYPKYLPIMDMAENLINDEGEFHLITNKEVFGTQSRTITNYWAQLALQPENFVVLNSVDAKKLKVDNGDKVLVTSTSNPKGEHLIAADESRPTIGKVKIVEGIRPGVVAISTHYGHWGYGARDIQIDGEVVKGEEVRGLGIHPNPLFRLDDNLRGTTLSDPIGGSASYYDTRVNIQRV

SEQ ID NO: 7 - DNA, 957 bp, Bacillus selenatarsenatis SF-1
atggagttaataagtgggtttgctgttggggttggattaacaatcttgatttatcgatcaaggctaaaaaggtttcatgatcatgaaccgaatgaacaggaacagaggattgtcaggctagtggaagactcaaaggatatcgtttactattatcaagtaaagccggaatttaaatttaaatatataagtccttctctggacctttatttgggtccaggggtcgtaaaagcggccatggaaaatccattcgactgttttgagcgttctcatcctgatgatgttgatcatttacataataaagtgacaggaaatctggattattcaaagcctatccttcagagatggctgggcccagatggccgctacctctggtttgaagaacatgcaagcccggtatatgaggatggacaactagttgctgtccagggaatcattcgaaatattgatgaaaaaatccaactgcagaaagacttggaatatcggatttaccatgatgccttaacaggaatctataatcgtcaatatttcgaacaaaaggcagcattttttaatataaaaaagaatagtcctattgcaatcattttgtgtgatttagataatttaaaaatgatgaatgattcatttggacataagcaaggggacaggctgttaattgaaacagcgaagctattaaaatccttctcttcggataatatttctgtctcaagaatcggaggtgatgaattcgccattctcctggctgataagaaagaagaatccgtgtccgaccttatcaattttatcgagtcatctattgccctttttaacatggacaaacaagagtataaaattgaaatatcaattggatacgcatactccaatcgctcgattgggaaaatggataatttgttatccatagcagatgcgaatatgtatagagttaaaaggcgtaaaaaggaatatacatacggcttataa

SEQ ID NO: 8 - protein, 318 aa, Bacillus selenatarsenatis SF-1
MELISGFAVGVGLTILIYRSRLKRFHDHEPNEQEQRIVRLVEDSKDIVYYYQVKPEFKFKYISPSLDLYLGPGVVKAAMENPFDCFERSHPDDVDHLHNKVTGNLDYSKPILQRWLGPDGRYLWFEEHASPVYEDGQLVAVQGIIRNIDEKIQLQKDLEYRIYHDALTGIYNRQYFEQKAAFFNIKKNSPIAIILCDLDNLKMMNDSFGHKQGDRLLIETAKLLKSFSSDNISVSRIGGDEFAILLADKKEESVSDLINFIESSIALFNMDKQEYKIEISIGYAYSNRSIGKMDNLLSIADANMYRVKRRKKEYTYGL

SEQ ID NO: 9 - DNA, 411 bp, Bacillus selenatarsenatis SF-1
atggatatttgggaggggtctgctggagtttgtttaaatgaacagggagagatcctcatggttcgccaggggaaaaaggacgaagaaaaactatgggcagtacctgctggcggtaaggaagacggagagacttttgagcagtgctgcatcagggaagtgctggaagagaccggatatgaagcagaaatcgtccgacagctttttgtgaaaactggcgtcttttttggagtggaagtcaaggttcagtacttcgagataaaaataataggaggagacctgcggattcaggaccctgatggattaatccatgaaattgcctggaaaaaggtgagtgagatgagcaaatcagattttggcttcaaagaagatagggagtttctgatgaagtttacgggcagccttcaagtataa

SEQ ID NO: 10 - protein, 136 aa, Bacillus selenatarsenatis SF-1
MDIWEGSAGVCLNEQGEILMVRQGKKDEEKLWAVPAGGKEDGETFEQCCIREVLEETGYEAEIVRQLFVKTGVFFGVEVKVQYFEIKIIGGDLRIQDPDGLIHEIAWKKVSEMSKSDFGFKEDREFLMKFTGSLQV

SEQ ID NO: 11 - DNA, 24 bp, Artificial Sequence
  Synthetic primer OPERON1F
ccagaaacagcaaagtccttgtcg

SEQ ID NO: 12 - DNA, 24 bp, Artificial Sequence
  Synthetic primer OPERON1R
gcagcttccctttcgcacaaagtt

SEQ ID NO: 13 - DNA, 30 bp, Artificial Sequence
  Synthetic primer ALLGGDEFFW
ggatccatggaataagcaaggccaggccaa

SEQ ID NO: 14 - DNA, 30 bp, Artificial Sequence
  Synthetic primer ALLGGDEFRV
ggatccttgccattctggttcacatggctg

SEQ ID NO: 15 - DNA, 30 bp, Artificial Sequence
  Synthetic primer UPALLGGDEFFW
gagctcatggaataagcaaggccaggccaa

SEQ ID NO: 16 - DNA, 30 bp, Artificial Sequence
  Synthetic primer UPALLGGDEFRV
gtcgacttgccattctggttcacatggctg

SEQ ID NO: 17 - DNA, 30 bp, Artificial Sequence
  Synthetic primer DOWNALLGGDEFFW
ccgcggatggaataagcaaggccaggccaa

SEQ ID NO: 18 - DNA, 30 bp, Artificial Sequence
  Synthetic primer DOWNALLGGDEFRV
gacgtcttgccattctggttcacatggctg

SEQ ID NO: 19 - protein, 5 aa, Artificial Sequence
  Synthetic peptide
GGDEF